(19) United States
(12) Patent Application Publication
(10) Pub. No.: US 2026/0118230 A1
(43) Pub. Date: Apr. 30, 2026

(54) Vehicle Cabin Gas Sensing System

(71) Applicant: Joyson Safety Systems Acquisition LLC, Auburn Hills, MI (US)

(72) Inventors: Salvatore Brauer, Royal Oak, MI (US); Caleb Breckenridge, Millington, MI (US); Leonard Cech, Brighton, MI (US)

(21) Appl. No.: 19/003,870

(22) Filed: Dec. 27, 2024

Related U.S. Application Data
(60) Provisional application No. 63/616,007, filed on Dec. 29, 2023.
(60) Provisional application No. 63/615,992, filed on Dec. 29, 2023.

(51) Int. Cl. G01N 1/14; B60H 3/00; G01N 15/075

(52) U.S. Cl. CPC G01N 1/14; B60H 3/0035; G01N 15/075

(57) ABSTRACT

A vehicle cabin gas sensing system includes a tube having a first end and a second end. The first end is coupled to a valve and the second end is disposed within a vehicle cabin. The system further includes a gas analysis chamber which is coupled to the valve and includes a gas analysis sensor. A pump is coupled to the gas analysis chamber and configured to move gas through the tube, the valve, and the gas analysis chamber. A controller includes a processor which is configured to start the pump, open the valve, and analyze the gas moved by the pump using the gas analysis sensor. By analyzing the composition of the gas, the vehicle cabin has sensing system is able to detect undesirable conditions in the vehicle cabin.

### CROSS REFERENCE TO RELATED APPLICATIONS

This application claims the benefit of U.S. Provisional Application No. 63/616,007 filed on Dec. 29, 2023, and claims the benefit of U.S. Provisional Application No. 63/615,992 filed on Dec. 29, 2023, the contents of which are incorporated herein by reference in their entirety.

### TECHNICAL FIELD

The present disclosure relates to safety and comfort devices for vehicles. In particular, the disclosure relates to a vehicle cabin gas sensing system for detecting and responding to various situations. Vehicles may include any type capable of being used for transportation, such as automobiles and aircraft, for example.

### BACKGROUND

Monitoring the interior cabin of a vehicle is important for the safety and comfort of vehicle occupants. For example, determining if an operator of the vehicle is intoxicated by alcohol or other substance can be lifesaving. Additionally, tracking malodors inside the vehicle cabin can help determine when the vehicle cabin would be uncomfortable for occupants. For example, in autonomous taxis or ride-sharing vehicles, many different occupants may use the vehicle throughout any given day and may bring pets, food, or other items into the vehicle that can impact the environment of the vehicle cabin.

One way to track alcohol intoxication is based on breath analysis. However, current systems for testing the breath of a vehicle occupant are invasive, expensive, and not aesthetically pleasing, leading to low adoption rates. Additionally, a typical alcohol breath analyzer does not analyze the breath sample for traces of other intoxicating chemicals or otherwise any other chemicals at all. Therefore, there is a need for a vehicle cabin gas sensing system that is capable of detecting many different chemicals throughout the vehicle cabin to allow for remedial activity, while being nearly or completely imperceptible to the vehicle occupants.

### SUMMARY

In various implementations, a vehicle cabin gas sensing system comprises a tube comprising a first end and a second end. The first end is coupled to a valve and the second end is disposed within a vehicle cabin. A gas analysis chamber comprises a gas analysis sensor and is coupled to the valve. A pump is coupled to the gas analysis chamber and configured to move gas through the tube, the valve, and the gas analysis chamber. The system comprises a controller comprising a processor and a memory, and the processor executes instructions stored in the memory causing the processor to start the pump, open the valve, and analyze the gas moved by the pump using the gas analysis sensor.

In some implementations, a vehicle cabin gas sensing system comprises a plurality of tubes each comprising a first end and a second end. Each of the first ends are coupled to a valve and each of the second ends are disposed within a vehicle cabin. The valve is capable of being opened or closed with respect to each of the plurality of tubes. A gas analysis chamber comprises a gas analysis sensor and is coupled to the valve. A pump is coupled to the gas analysis chamber and configured to move gas through the plurality of tubes, the valve, and the gas analysis chamber. The system comprises a controller comprising a processor and a memory, and the processor executes instructions stored in the memory causing the processor to start the pump, open the valve with respect to one of the plurality of tubes and close the valve with respect to each of the remaining tubes of the plurality of tubes, and analyze the gas moved by the pump using the gas analysis sensor.

### BRIEF DESCRIPTION OF THE DRAWINGS

The drawings are merely exemplary to illustrate steps, structure, and certain features that can be used singularly or in combination with other features. The disclosure should not be limited to the implementations shown.

FIG. 1 is a side perspective view of a vehicle cabin and a first implementation of a vehicle cabin gas sensing system.

FIG. 2 is a side perspective view of a vehicle cabin and another implementation of a vehicle gas sensing system.

FIG. 3A is a front view of a vehicle seat from FIG. 2 with an occupant seated on the seat.

FIG. 3B is another front view of a vehicle seat from FIG. 2 with an occupant seated on the seat.

FIG. 3C is another front view of a vehicle seat from FIG. 2 with an occupant seated on the seat.

### DETAILED DESCRIPTION

The present disclosure relates to safety and comfort devices for vehicles. The devices, assemblies, and methods disclosed herein provide for a vehicle cabin gas sensing system for any type of vehicle capable of transporting occupants. The vehicle cabin gas sensing system moves gas through the system and detects the composition of that gas using a gas analysis sensor. A controller comprising a processor and a memory compares the composition of the gas to predefined thresholds set for specific constituents in order to determine the condition of the vehicle cabin environment. When specific constituents in the gas are detected above system defined thresholds, the vehicle cabin gas sensing system can alert the vehicle operator (either local or remote) and/or take certain remedial actions.

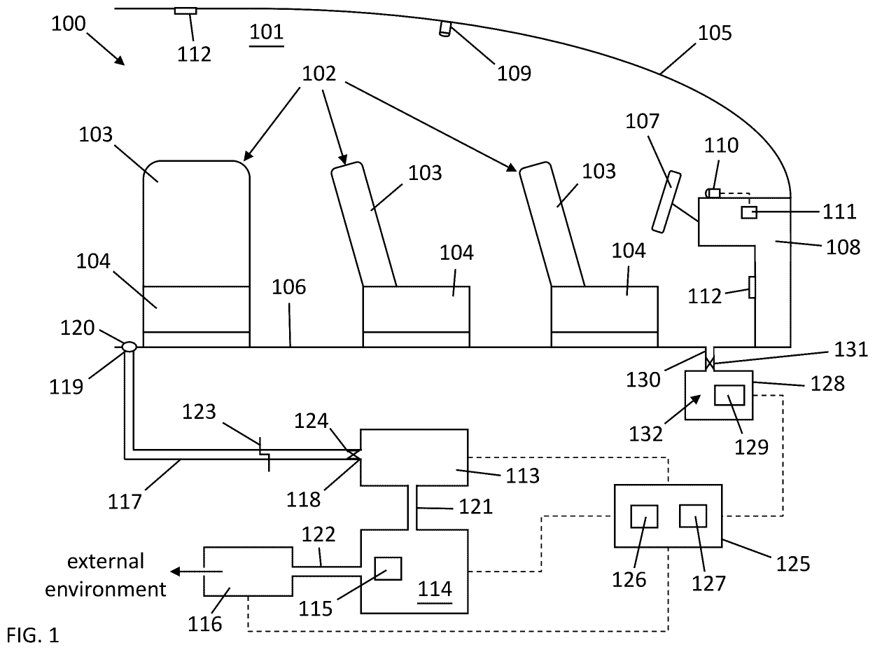

As shown in FIG. 1, a first implementation of a vehicle cabin gas sensing system 100 includes a vehicle having a vehicle cabin 101 comprising at least one seat 102. In the example shown in FIG. 1, the vehicle cabin 101 includes three seats 102, two forward-facing (facing the front of the vehicle cabin 101) and one side-facing (perpendicular to the forward-facing seats). However, in other implementations, the vehicle cabin 101 may contain any number of seats in any desired orientation as determined by the vehicle manufacturer. The seats 102 each comprise a seat back 103 and a seat bottom 104.

The vehicle cabin 101 further comprises a dashboard 108 to which a steering wheel 107 is mounted for operation of the vehicle by an operator. A driver monitoring system (DMS) 110 is coupled to the dashboard 108. The DMS 110 may include a camera with a field of view that encompasses the operator's head and is capable of tracking the attention of the operator, such as is described in U.S. Pat. No. 9,041,789, which is herein incorporated by reference in its entirety and is being filed herewith in an Appendix. The DMS 110 is electronically coupled, either wired or wirelessly, to a DMS computer 111. The DMS computer 111 may include a memory and a processor for executing instructions stored on the memory, wherein the instructions cause the processor to track the attention of the operator and determine if the operator is impaired. Additionally, an occupant monitoring system (OMS) 109 is coupled to a ceiling 105 of the vehicle cabin 101. The OMS 109 may include a camera with a field of view that encompasses the occupants of the vehicle cabin 101 other than the operator of the vehicle, however the field of view of the OMS 109 may also include the operator, such as is described in U.S. Pat. Nos. 7,406,181 and 10,611,335. U.S. Pat. No. 7,406,181 is herein incorporated by reference in its entirety and is being filed herewith in an Appendix. The OMS 109 may also be electronically coupled, either wired or wirelessly, to the DMS computer 111. As used herein, “camera” is meant to refer to any imaging device capable of generating an image processable by a computer, including 2D cameras, 3D cameras (e.g., 3D TOF), radar, and lidar.

The vehicle cabin 101 may also comprise one or more environmental sensors 112. For example, in FIG. 1, the vehicle cabin 101 includes two environmental sensors 112, one coupled to the ceiling 105 and the other coupled to the dashboard 108. In some implementations, the environmental sensors 112 may be chosen from temperature sensors, humidity sensors, pressure/force sensors, vibration sensors, acceleration sensors, or combinations thereof. However, in other implementations, the environmental sensors may be chosen from any sensor that provides information about the internal environment of the vehicle cabin 101.

The vehicle cabin gas sensing system 100 comprises a valve 113 and a tube 117 comprising a first end 118 and a second end 119. The first end 118 is coupled to the valve 113. The second end 119 is disposed within the vehicle cabin 101. A portal 120 may be coupled to the second end 119. The portal 120 may comprise a decorative trim component and/or a particulate filter, so long as the second end 119 remains in fluid communication with the vehicle cabin 101. For example, as shown in FIG. 1, the second end 119 and portal 120 are positioned adjacent a vehicle floor 106, such as in a hole in the vehicle floor 106 providing access to the vehicle cabin 101.

The tube 117 may be made from any material as required by vehicle characteristics. For example, the tube 117 may comprise PVC or other rigid plastic tubing, PEX or other flexible plastic tubing, or rigid or flexible metal tubing. The length of the tube 117 and the routing of the tube 117 throughout the vehicle may vary depending on vehicle characteristics. For example, the valve 113 and the rest of the vehicle cabin gas sensing system 100 (described below) may be located anywhere within the vehicle as required, for example under the floor 106 (e.g., FIG. 1, not drawn to scale), in the ceiling 105, in the dashboard 108, or elsewhere in the vehicle.

The valve 113 comprises a solenoid 124 located adjacent the first end 118 of the tube 117 and is integrally part of the valve 113. The valve 113 is coupled to a gas analysis chamber 114 via a conduit 121. The gas analysis chamber 114 comprises a gas analysis sensor 115 and is further coupled to a pump 116 via a conduit 122. The pump 116 may be a fan, a vacuum, or any other device capable of pulling gas through the vehicle cabin gas sensing system 100 by producing a negative pressure thereby pulling gas from the vehicle cabin 101 through to the gas analysis chamber 114 and ultimately out to an external environment (e.g., back into the vehicle cabin 101 or external of the vehicle). The conduits 121, 122 may be similar to the tube 117, or the valve 113, gas analysis chamber 114, and pump 116 may all be integrated into one common housing or other structure, so long as fluid communication is possible between the components. The gas analysis chamber 114 may be defined by a housing or other structure, such as a plastic or metal housing, that allows gas to flow over or through the gas analysis sensor 115.

One representative gas analysis sensor 115 is based on transmission spectroscopy using a broad spectral band light source which emits light into the gas analysis chamber 114. An example of a transmission spectrometer sensor is a Michelson interferometer (other spectrometers could also be used, such as a Fabry-Perot or grating spectrometer). The Michelson interferometer comprises an optical system located adjacent to or spaced apart from the broad spectral band light source. The Michelson interferometer further comprises a fixed mirror, a movable mirror, and a partially reflective mirror. By controlling the movable mirror as a function of time, the system sweeps through all individual wavelengths within the broad spectral band light source, typically comprising visible, near infrared, and mid-infrared wavelengths. A digital detector, which detects the received light at each wavelength, produces a voltage versus time signal which is then processed through a digital Fourier transform after each full mirror sweep to produce an absorbance spectrum (i.e., amplitude versus wavelength). Since each chemical molecule has a unique deterministic absorbance spectrum, digital algorithms (e.g., principal least squares, principal components analysis, chemometrics, Al, etc.) can be used to determine the chemicals within the sample and estimate their concentrations within the sample. In other implementations, other gas analysis sensors could be used. For example, electrochemical MEMs sensors operate by having unique cathode/anode chemistries for multiple target chemicals that generate a deterministic voltage as a function of the concentration in a gas sample.

The valve 113 may be an electromechanical solenoid valve as known in the art and used in numerous industries including industrial, medical, and automotive for gas and/or fluid control. In some implementations, one or more discrete solenoid microvalves or a micro-manifold (several valves integrated into a single component) are used. For example, the valve 113 may be made by companies such as miniValve (https://minivalve.com) or the Lee Company (https://www.theleeco.com). Such valves are connected to input and output ports (e.g., connected to airtight tubes) and are electromagnetically controllable to open/close quickly and securely over potentially millions of cycles. A general method to open/close such valves includes providing a positive voltage/current to an electrical coil of a solenoid which moves a plunger to the open or closed position as long as the voltage/current is applied. Such solenoid plungers may also include a mechanical resistance system (e.g., a spring) to assure that the plunger moves back to the correct mechanical state after the voltage/current is turned off. Other components can also be integrated into such valves, for example filters (e.g., particulates, humidity, or chemical specific) and/or one-way check valves depending on the requirements of the vehicle cabin gas sensing system 100.

A controller 125 is electronically coupled to the valve 113, gas analysis sensor 115, and pump 116 in order to control the operation of vehicle cabin gas sensing system 100 (electrical coupling represented by dashed lines in FIG. 1, for example). The controller 125 may be directly coupled via wired connections or wirelessly connected through a wireless protocol, such as WIFI or BLUETOOTH. The controller 125 comprises a processor 126 and a memory 127. The processor 126 executes instructions stored on the memory 127 to open the valve 113 (e.g., causing the valve 113 to open/activate the solenoid 124), operate the gas analysis sensor 115, and turn on the pump 116. Therefore, when the vehicle cabin gas sensing system 100 is operating, the pump 116 pulls gas out of the vehicle cabin 101 through the tube 117, the valve 113, the conduit 121, the gas analysis chamber 114, the conduit 122, the pump 116, and out into the external environment. During this time, the gas analysis sensor 115 may continually or intermittently (e.g., every few seconds or minutes) analyze the gas for its composition.

The memory 126 may also store predefined threshold limits for gas concentration of various gas constituents so that the controller 125 may identify when a hazardous or otherwise undesirable condition exists within the vehicle cabin 101. The controller 125 may continuously operate the vehicle cabin gas sensing system 100 or it may operate the system 100 intermittently. For example, the controller 125 may start the pump 116, open the valve 113, and analyze gas moved by the pump 116 using the gas analysis sensor 115 for a period of one minute out of every ten minutes. When not operating, the vehicle cabin gas sensing system 100 may sit idle with no gas moving through the system 100. Optionally, a one-way check valve 123, passively operable by pressure, may be disposed anywhere within the tube 117 to ensure gas only flows from the vehicle cabin 101 to the valve 110 and not vice versa.

A variety of gas constituents can be identified by the gas analysis sensor 115. For example, any volatile organic compound, such as alcohols (e.g., ethanol, methanol, isopropyl, propane, butane, etc.), carbon dioxide, carbon monoxide, ketones, aldehydes, carboxylic acids, aromatics, urea, ammonia, smoke (e.g., cigarette or vape), and many others can indicate a variety of conditions within the vehicle cabin 101. If alcohol is detected, it is possible a vehicle operator is intoxicated and unable to operate the vehicle safely. If urea or smoke are detected, for example, the vehicle cabin 101 may have an undesirable smell that would lower occupant comfort, such as from pet waste.

To counteract some of these issues, a reservoir 128 containing a neutralizing agent 132 may be included within the vehicle. The reservoir 128 may include a pump 129 electronically coupled to the controller 125 and instructions stored in the memory 127 may cause the processor 126 to operate the pump 129 to pump the neutralizing agent 132 into the vehicle cabin 101. The neutralizing agent 132 may be a pleasant-smelling air freshener or other agent that chemically reacts with undesirable or unsafe gases or alternatively has a positive physiological effect on occupants. If urea or smoke are detected, for example, the pump 129 may open a solenoid 131 allowing the neutralizing agent 132 to pass through a conduit 130 and into the vehicle cabin 101.

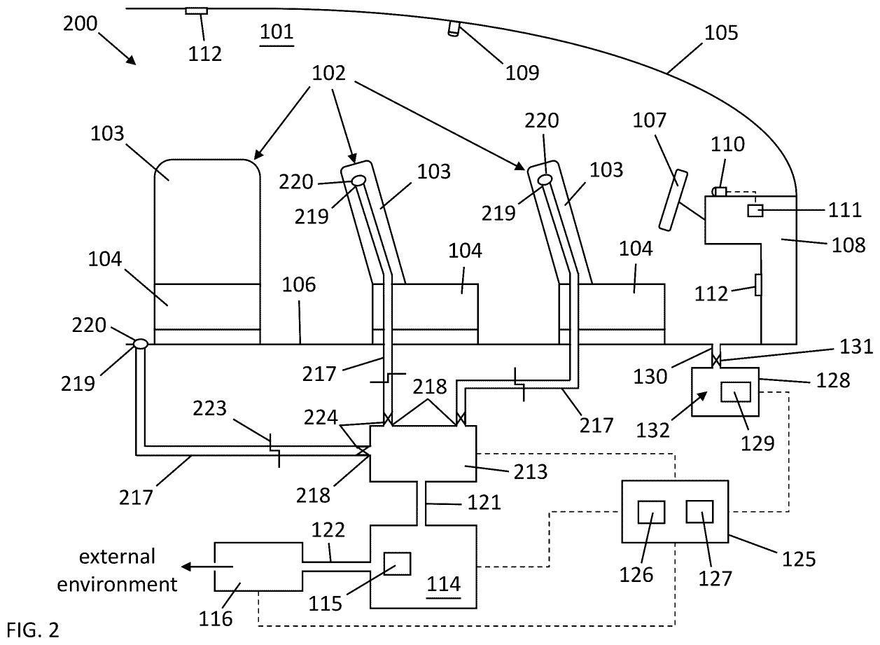

Referring now to FIG. 2, a second implementation of a vehicle cabin gas sensing system 200 comprises a plurality of tubes 217. As shown in FIG. 2, a total of three tubes 217 are included, however in other implementations any number of tubes may be used depending on the vehicle cabin environment and the desired performance of the vehicle cabin gas sensing system 200. The system 200 is similar to the system 100, however in this example a valve 213 comprises multiple connections to a plurality of tubes 217, each of the plurality of tubes 217 comprising a first end 218 coupled to the valve 213 and a second end 219 (and portal 220) disposed within the vehicle cabin 101. Each of the plurality of tubes 217 may comprise a one-way check valve 223, passively operable by pressure, disposed anywhere within the tubes 217 to ensure gas only flows from the vehicle cabin 101 to the valve 213 and not vice versa. Similarly, the valve 213 comprises a plurality of solenoids 224, each operable with respect to one of the plurality of tubes 217. This allows the valve 213 to selectively open only one of the plurality of tubes 217 at any given time, therefore allowing the vehicle cabin gas sensing system 200 to diagnose the status of the environment in localized regions of the vehicle cabin 101.

As an example, the controller 125 may instruct the valve 213 to open with respect to one of the plurality of tubes 217 and close with respect to each of the remaining tubes 217 of the plurality of tubes 217 (e.g., open/activate one solenoid 224 and close all others). The controller 125 can then instruct the pump 116 to begin pulling gas through the system 200 and instruct the gas analysis sensor 115 to begin analyzing the composition of the gas, similar to the above description for the system 100. In this way, the gas analysis sensor 115 will only be analyzing gas that is associated with a localized region of the vehicle cabin 101 at any given time, rather than the entire vehicle cabin 101, therefore allowing the controller 125 to identify undesirable conditions (e.g., intoxicated operator or pet waste).

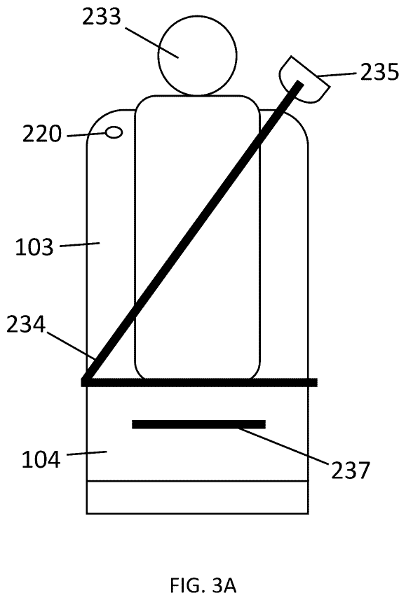

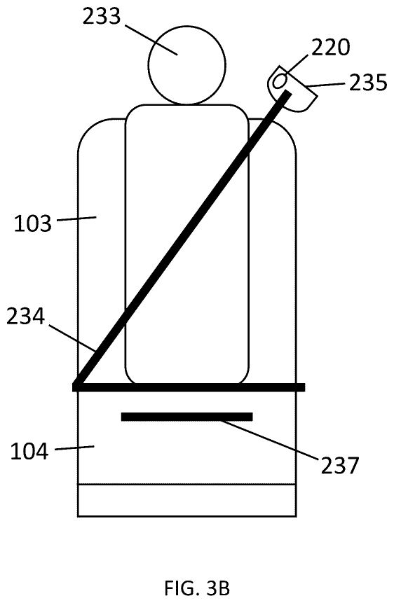

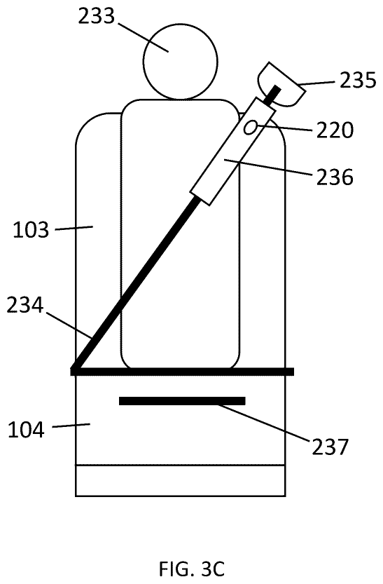

As shown in FIG. 2, two of the plurality of tubes 217 extend within two of the seats 102. The plurality of tubes 217 can be routed through the vehicle floor 106 and through the seat bottoms 104 and seat backs 103 such that they are not visible to vehicle occupants. FIGS. 3A-3C show specific examples of locating the second end 219 and portal 220 of one of the plurality of tubes 217 in one of the seats 102. For example, as shown in FIG. 3A, the portal 220 is located near the top of the seat back 103 near the head and shoulders of an occupant 233 sitting on the seat bottom 104. A seat-based occupant classification system 237 may be installed in the seat bottom 104 and/or seat back 103 to detect the occupant 233. If no occupant 233 is detected, the controller 125 will not open the valve 213 with respect to the relevant tube 217. An example occupant classification system 237 is described in U.S. Pat. No. 10,926,662, which is herein incorporated by reference in its entirety and is being filed herewith in an Appendix.

The second end 219 and portal 220 may be located in other locations with respect to the seat 102. As shown in FIG. 3B, the second end 219 and portal 220 are located on or adjacent a seatbelt shoulder anchor 235 through which a seatbelt webbing 234 is routed. Alternatively, as shown in FIG. 3C, the second end 219 and portal 220 may be located on or within a seatbelt gas sensing assembly 236 that is coupled to the seatbelt webbing 234 and positioned adjacent the head and shoulders of the occupant 233. In this way, the system 200 is capable of identifying whether a specific vehicle occupant is intoxicated with alcohol, other intoxicating substance, or otherwise impaired in any way. Thus, the vehicle cabin gas sensing system 200 is able to increase safety in the vehicle.

What is claimed is:

1. A vehicle cabin gas sensing system comprising:
a tube comprising a first end and a second end, the first end coupled to a valve and the second end disposed within a vehicle cabin;
a gas analysis chamber comprising a gas analysis sensor, wherein the valve is coupled to the gas analysis chamber;
a pump coupled to the gas analysis chamber and configured to move gas through the tube, the valve, and the gas analysis chamber; and
a controller comprising a processor and a memory;
wherein the processor executes instructions stored in the memory, the instructions causing the processor to start the pump, open the valve, and analyze the gas moved by the pump using the gas analysis sensor.

2. The vehicle cabin gas sensing system of claim 1, wherein the second end of the tube is disposed on or within a vehicle seat.

3. The vehicle cabin gas sensing system of claim 1, wherein the second end of the tube is disposed adjacent a seatbelt shoulder anchor.

4. The vehicle cabin gas sensing system of claim 1, wherein the second end of the tube is disposed within a seatbelt gas sensing assembly.

5. The vehicle cabin gas sensing system of claim 1, wherein the second end of the tube is disposed adjacent a floor of the vehicle cabin.

6. The vehicle cabin gas sensing system of claim 1, further comprising one or more environmental sensors disposed within the vehicle cabin.

7. The vehicle cabin gas sensing system of claim 6, wherein the one or more environmental sensors may be chosen from temperature sensors, humidity sensors, pressure/force sensors, vibration sensors, acceleration sensors, or combinations thereof.

8. The vehicle cabin gas sensing system of claim 1, wherein the tube is closable by a one-way check valve disposed within the tube.

9. The vehicle cabin gas sensing system of claim 8, wherein the one-way check valve is passively operated by pressure.

10. The vehicle cabin gas sensing system of claim 1, wherein opening the valve comprises the instructions causing the valve to activate a solenoid.

11. The vehicle cabin gas sensing system of claim 1, further comprising a reservoir containing a neutralizing agent.

12. The vehicle cabin gas sensing system of claim 11, wherein the reservoir comprises a reservoir pump, wherein the processor executes instructions stored on the memory to cause the reservoir pump to pump the neutralizing agent into the vehicle cabin.

13. A vehicle cabin gas sensing system comprising:
a plurality of tubes each comprising a first end and a second end, each of the first ends coupled to a valve and each of the second ends disposed within a vehicle cabin, the valve capable of being opened or closed with respect to each of the plurality of tubes;
a gas analysis chamber comprising a gas analysis sensor, wherein the valve is coupled to the gas analysis chamber;
a pump coupled to the gas analysis chamber and configured to move gas through the plurality of tubes, the valve, and the gas analysis chamber; and
a controller comprising a processor and a memory;
wherein the processor executes instructions stored in the memory, the instructions causing the processor to start the pump, open the valve with respect to one of the plurality of tubes and close the valve with respect to each of the remaining tubes of the plurality of tubes, and analyze the gas moved by the pump using the gas analysis sensor.

14. The vehicle cabin gas sensing system of claim 13, wherein at least one of the second ends of the plurality of tubes is disposed on or within a vehicle seat.

15. The vehicle cabin gas sensing system of claim 13, wherein at least one of the second ends of the plurality of tubes is disposed adjacent a seatbelt anchor.

16. The vehicle cabin gas sensing system of claim 13, wherein at least one of the second ends of the plurality of tubes is disposed with a seatbelt gas sensing assembly.

17. The vehicle cabin gas sensing system of claim 13, wherein at least one of the second ends of the plurality of tubes is disposed adjacent a floor of the vehicle cabin.

18. The vehicle cabin gas sensing system of claim 13, further comprising one or more environmental sensors disposed within the vehicle cabin.

19. The vehicle cabin gas sensing system of claim 18, wherein the one or more environmental sensors may be chosen from temperature sensors, humidity sensors, pressure/force sensors, vibration sensors, acceleration sensors, or combinations thereof.

20. The vehicle cabin gas sensing system of claim 13, wherein each of the plurality of tubes is closable by a one-way check valve disposed within each of the plurality of tubes.

21. The vehicle cabin gas sensing system of claim 20, wherein the each of the one-way check valves is passively operated by pressure.

22. The vehicle cabin gas sensing system of claim 13, wherein opening the valve with respect to one of the plurality of tubes and closing the valve with respect to each of the remaining tubes of the plurality of tubes comprises the instructions causing the valve to activate a plurality of solenoids.

23. The vehicle cabin gas sensing system of claim 13, further comprising a reservoir containing a neutralizing agent.

24. The vehicle cabin gas sensing system of claim 23, wherein the reservoir comprises a reservoir pump, wherein the processor executes instructions stored on the memory to cause the reservoir pump to pump the neutralizing agent into the vehicle cabin.

25. The vehicle cabin gas sensing system of claim 13, further comprising a camera-based occupant monitoring system, wherein the controller uses an output from the camera-based occupant monitoring system to open the valve with respect to one of the plurality of tubes and close the valve with respect to each of the remaining tubes of the plurality of tubes.

26. The vehicle cabin gas sensing system of claim 13, further comprising a seat-based occupant classification system, wherein the controller uses an output from the seat-based occupant classification system to open the valve with respect to one of the plurality of tubes and close the valve with respect to each of the remaining tubes of the plurality of tubes.
